(19) United States
(12) Patent Application Publication
(10) Pub. No.: US 2014/0107050 A1
(43) Pub. Date: Apr. 17, 2014

(54) INCREASING BIOAVAILABILITY OF N-COUMAROYLDOPAMINE THROUGH CO-ADMINISTRATION WITH A CATECHOL-O-MEHTYLTRANSFERASE (COMT) INHIBITOR

(71) Applicant: Redefine Nutrition, LLC, Alpharetta, GA (US)

(72) Inventors: Kyung S. Kim, Cumming, GA (US); Bryan J. Krause, Cumming, GA (US)

(73) Assignee: Redefine Nutrition, LLC, Alpharetta, GA (US)

(21) Appl. No.: 14/053,172

(22) Filed: Oct. 14, 2013

Related U.S. Application Data
(60) Provisional application No. 61/713,863, filed on Oct. 15, 2012.

(51) Int. Cl. A61K 31/165; A61K 31/522; A61K 31/137; A61K 45/06; A61K 31/197; A61K 31/353; A61K 31/506

(52) U.S. Cl. CPC A61K 31/165; A61K 31/353; A61K 31/522; A61K 31/506; A61K 45/06; A61K 31/197; A61K 31/137
USPC 514/27; 514/622; 514/456; 514/292; 514/570; 514/533; 514/263.31; 514/256; 514/561

(57) ABSTRACT

The present invention is directed to a composition including N-Coumaroyldopamine and a catechol-o-methyltransferase (COMT) inhibitor for promoting weight loss in a user.

### CROSS-REFERENCE TO RELATED APPLICATION

This application claims the benefit of U.S. Provisional Patent Application Ser. No. 61/713,863, filed Oct. 15, 2012, which is incorporated herein in its entirety by reference.

### FIELD OF THE INVENTION

Generally, the present invention is directed to dietary supplements. More specifically, the present invention is related to compositions containing N-coumaryldopamine and methods of use thereof which improve body fat metabolism, weight loss, and increase aerobic capacity.

### BACKGROUND OF THE INVENTION

Obesity and related heath complications are continuing problems among the developed nations. According to the American Heart Association, over 60% of men and nearly 50% of women in the United States are overweight. Even worse, approximately 13% of men and 18% of women are obese. Increased weight and obesity have direct links to a number of health risks and diseases, such as elevated blood pressure, high cholesterol, heart disease, joint disease, and type II diabetes, to name a few.

To combat this problem, many diets, exercise programs, pharmaceuticals, and herbal supplements have been developed. However, some previously marketed supplements are no longer available due to adverse side effects. Thus, there is a need for supplements which promote weight loss and lead to improved exercise performance without adverse side effects.

Phytochemicals are naturally occurring chemical compounds in plants. Some phytochemicals exhibit an antioxidant and/or therapeutic effect. N-coumaroyldopamine is a naturally occurring compound found in various plants, including the cocoa plant (Theobroma cacao). Recent in vitro studies indicate N-coumaroyldopamine acts as a beta-adrenoreceptor agonist, see, e.g., Park, G. (2005) N-Coumaroyldopamine and N-caffeoyldopamine increase cAMP via beta 2-adrenoreceptors in myelocytic U937 cells, FASEB Journal, 19, 497-502. Beta-adrenoreceptor agonists have the potential to aid in bronchodilation, weight loss, and central nervous system stimulation by increasing the production of cAMP.

Use of N-coumaroyldopamine as a natural health enhancer or weight loss supplement is limited due to its rapid metabolism by catechol-o-methyltransferase (COMT). Thus, COMT inhibitors have the potential to inhibit N-coumaroyldopamine metabolism, thereby prolonging the half-life upon ingestion. Exemplary COMT inhibitors include quercetin and chlorogenic acid, which inhibit COMT with a half maximal inhibitory concentration (IC₅₀) of about 0.5 μM and about 1.3-1.4 μM, respectively (see, e.g., van Duursen, M. B. M. et al. (2004) Phytochemicals inhibit catechol-o-methyltransferase activity in cytosolic fractions from healthy human mammary tissues: Implications for catechol estrogen-induced DNA damage. Toxicological Sciences, 81 (2), 316-324; Zhu, B. T. et al. (2009) Inhibition of human catechol-o-methyltransferase (COMT)-mediated o-methylation of catechol estrogens by major polyphenolic components present in coffee, Journal of Biochemistry and Molecular Biology, 113(1-2), 65-74). Epigallocatechin gallate (EGCG) demonstrates a higher relative degree of COMT inhibition, with an IC₅₀ of about 0.2 μM (Lu, H. et al. (2003) Enzymology of methylation of tea catechins and inhibition of catechol-o-methyltransferase by (—)-epigallocatechin gallate, Drug Metabolism and Disposition, 31, 572-579).

Accordingly, there is a need for a composition which provides increased N-coumaroyldopamine bioavailability to facilitate body fat mobilization, weight loss, and exercise performance. It is to solving this and other problems the present invention is directed.

### SUMMARY OF THE INVENTION

The present invention is directed to a composition including N-coumaroyldopamine. According to one aspect of the present invention, a composition comprises N-coumaroyldopamine, a catechol-o-methyltransferase inhibitor, and, optionally, one or more excipients. The catechol-o-methyltransferase inhibitor includes, but is not limited to, epigallocatechin gallate, quercetin, harmine, rutin, caffeic acid, chlorogenic acid, rosmarinic acid, or any combination thereof. Yet in another aspect, the catechol-o-methyltransferase inhibitor is a catechin. In another aspect, a composition comprises N-coumaroyldopamine, epigallocatechin gallate, and one or more excipients.

Still yet in another aspect, a method to facilitate body fat mobilization, weight loss, or exercise performance in a user comprises administering a composition comprising N-coumaroyldopamine and a catechol-o-methyltransferase inhibitor in a physiologically effective amount to decrease body fat, enhance weight loss, or improve exercise performance.

It is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the present invention. It is important, therefore, that the claims be regarded as including such equivalent constructions insofar as they do not depart from the spirit and scope of the present invention.

Other advantages and capabilities of the invention will become apparent from the following description taken in conjunction with the examples showing aspects of the present invention.

### DETAILED DESCRIPTION OF THE INVENTION

For a fuller understanding of the nature and desired objects of this invention, reference should be made to the above and following detailed description. The present invention is directed to a composition comprising N-coumaroyldopamine and a catechol-o-methyltransferase (COMT) inhibitor. In another aspect, the present invention is directed to methods of use related to compositions comprising N-coumaroyldopamine and a COMT inhibitor. We discovered that N-coumaroyldopamine compositions containing the COMT inhibitor increase N-coumaroyldopamine bioavailability. In yet another aspect, a composition for promoting weight loss includes N-coumaroyldopamine, epigallocatechin gallate (EGCG), and one or more excipients.

“Percent purity,” “% purity,” “percent pure,” or “% pure” refer to the mass of a pure substance divided by the total mass of a compound or composition, multiplied by 100. Typically, “mass” is measured in grams (g). For example, a substance having a total mass of 100 g, which includes 80 g of substance A, is 80% pure. The remaining 20%, accordingly, typically comprise impurities and/or side reaction products.

“Percent by weight” and “% by weight” refer to the weight of a pure substance divided by the total weight of a compound or composition, multiplied by 100. Typically, “weight” is measured in g. For example, a composition with a total weight of 100 g, which includes 25 g of substance A, will include substance A in 25% by weight.

### N-Coumaroyldopamine

N-coumaroyldopamine, as represented by structure (3) below, occurs as a natural phytochemical in various plants, including the cocoa plant (Theobroma cacao). Moreover, N-coumaroyldopamine can be synthetically-derived by any suitable protocol. For example, a general reaction scheme includes combining p-coumaric acid, represented by structure (2), with dopamine hydrobromide, shown in structure (1). Amide bond formation, under appropriate reaction conditions, provides the N-coumaroyldopamine (3) product.

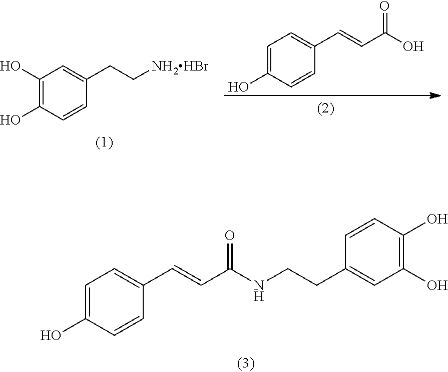

Naturally occurring N-coumaroyldopamine is present in trace amounts in various plants. Thus, natural plant extraction provides limited quantities of N-coumaroyldopamine. Moreover, N-coumaroyldopamine can have a low % purity. Synthetic pathways can provide N-coumaroyldopamine in increased yield and purity. In one aspect, N-coumaroyldopamine contained in the composition in accordance with the present invention is from about 80% to about 99% pure. In another aspect, N-coumaroyldopamine contained in the composition in accordance with the present invention is from about 90% to about 98% pure. Yet, in another aspect, N-coumaroyldopamine contained in the composition in accordance with the present invention is in any range between about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% pure. For example, in one aspect, the N-coumaroyldopamine is between about 50% and about 70% pure. In another aspect, the N-coumaroyldopamine is between about 67% and about 85% pure. Yet in another aspect, the N-coumaroyldopamine is between about 80% and about 99% pure. Still yet, in another aspect, the N-coumaroyldopamine is between about 85% and about 98% pure.

### Catechol-O-Methyltransferase (COMT) Inhibitor

As indicated above, a COMT inhibitor, when present in an N-coumaroyldopamine composition, substantially inhibits N-coumaroyldopamine metabolism. Absent COMT inhibition, N-coumaroyldopamine is metabolized very rapidly by the enzyme COMT. N-coumaroyldopamine's route of chemical inactivation via COMT can be inhibited with a COMT enzyme inhibitor. Examples of suitable naturally-derived COMT inhibitors include, but are not limited to: quercetin, harmine, rutin, caffeic acid, chlorogenic acid, rosmarinic acid, epigallocatechin gallate (EGCG), or any combinations thereof. EGCG is an ester of epigallocatechin and gallic acid and is the most abundant catechin naturally occurring in green tea. Thus, the COMT inhibitor can be any catechin. Extraction of a COMT inhibitor can be conducted by any methods known to those in the art.

A COMT inhibitor utilized in the composition of the present invention is at least about 50% pure. In another aspect, a COMT inhibitor utilized in the composition of the present invention has a purity in a range between about 40% and about 60% pure. Yet, in yet another aspect, a COMT inhibitor utilized in the composition of the present invention has a purity in a range between about 50% to about 90% pure. Still yet, in another aspect, a COMT inhibitor utilized in the composition of the present invention has a purity in any range between about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% pure. For example, in one aspect, the COMT inhibitor is between about 50% and about 70% pure. In another aspect, the COMT inhibitor is between about 67% and about 85% pure. Yet in another aspect, the COMT inhibitor is between about 80% and about 99% pure. Still yet, in another aspect, the COMT inhibitor is between about 85% and about 98% pure.

Increasing purity of a COMT inhibitor can be accomplished by any suitable method known to those in the art, including, but not limited to, extraction, vacuum distillation, re-crystallization, sublimation, drying, chromatography, or any combination thereof.

### Combination and Use of N-Coumaroyldopamine and a COMT Inhibitor

The composition including N-Coumaroyldopamine and a COMT inhibitor can be formulated into any dosage forms using commonly known manufacturing methods. The dosage form can be, but is not limited to, a pill, a tablet, a capsule, granules, a powder, a drink mix, an aqueous solution, a suspension, a beverage, a confectionary syrup, a chewable tablet, or any combination thereof.

The N-coumaroyldopamine and COMT inhibitor can be first prepared individually. The N-coumaroyldopamine and COMT inhibitor can be combined with one or more active ingredient, inactive ingredient, and/or suitable pharmaceutical excipient. Illustrative excipients include, but are not limited to, antioxidants, buffers, preservatives, thickening agents, colorants, bacteriostatic agents, stabilizers, or any combination thereof. The preparation of such compositions, including suitable excipients, will be known to those of skill in the art in light of the present disclosure, and as exemplified by Remington: The Science and Practice of Pharmacy, 21st Edition (2005) Lippincott Williams & Wilkins, which is incorporated herein in its entirety by reference. Generally, a given excipient, if present, will be present in an amount of about 0.001% to about 95%, about 0.01% to about 80%, about 0.02% to about 25%, or about 0.3% to about 10% by weight.

Illustrative antioxidants for use in the present invention include, but are not limited to, alpha tocopherol, ascorbic acid, acorbyl palmitate, butylated hydroxyanisole, butylated hydroxytoluene, potassium metabisulfite, propionic acid, propyl gallate, sodium ascorbate, sodium bisulfite, sodium metabisulfite, sodium sulfite, or any combination thereof. Illustrative chelating agents include, but are not limited to, citric acid monohydrate, disodium edetate, dipotassium edetate, edetic acid, fumaric acid, malic acid, phosphoric acid, sodium edetate, tartaric acid, trisodium edetate, or any combination thereof.

Suitable buffers include any agents that reduce pH changes. Illustrative classes of buffering agents include, but are not limited to, a salt of a Group IA metal including, for example, a bicarbonate salt of a Group IA metal, a carbonate salt of a Group IA metal, an alkaline or alkali earth metal buffering agent, an aluminum buffering agent, a calcium buffering agent, a sodium buffering agent, a magnesium buffering agent, or any combination thereof. Suitable buffering agents include carbonates, phosphates, bicarbonates, citrates, borates, acetates, phthalates, tartrates, succinates of any of the foregoing, for example sodium or potassium phosphate, citrate, borate, acetate, bicarbonate and carbonate, or any combination thereof. Non-limiting examples of suitable buffering agents include aluminum-magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometarnol, or any combination thereof. Furthermore, combinations or mixtures of any two or more of the above mentioned buffering agents can be used in the pharmaceutical compositions described herein.

Suitable preservatives include, but are not limited to, benzyalkonium chloride, benzethonium chloride, benzyl alcohol, bronopol, cetrimide, cetylpuridinium chloride, chorohexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, thimerosal, butyl paraben, methyl paraben, propyl paraben, benzoic acid, potassium sorbate, sodium benzoate, sodium propionate, sorbic acid, or any combination thereof.

Optional thickening agents include cellulose and cellulose derivatives including, but not limited to, cellulose acetate, microcrystalline cellulose, powdered cellulose, cellulose acetate phthalate, hydroxyethyl cellulose, silicified microcrystalline cellulose, hydroxypropyl cellulose, hydroxyethylmethyl cellulose, low-substituted hydroxypropyl cellulose, carboxymethylcellulose, carboxymethylcellulose calcium, hypromellose acetate succinate, hypromellose phthalate and ethylcellulose. Suitable natural polymers include acacia, tragacanth, xanthan gum, sodium alginate, and carrageenan.

Illustrative colorants include, but are not limited to, Red Ferric Oxide, Yellow Ferric Oxide, titanium dioxide, carbon black, or any combination thereof. Natural colorants include, but are not limited to, chlorophyll, anattenes, beta-carotene, alizarin, indigo, flavones (e.g. riboflavin, rutin, hesperidin, and quercetin), saffron, cudbear, red saunders, Tyrian, cochineal, or combinations thereof. Synthetic colorants include, but are not limited to, mauveine, nitroso-dyes, nitro-dyes, azo-dyes, oxazines, thiazines, pyrazolones, xanthenes, indigoids, anthraquinones, acridines, rosanilines, phthaleins, quinolones, or any combination thereof.

The foregoing excipients, which are discussed in Remington, The Science and Practice of Pharmacy, 21^{st} ed., (2005) Lippincott Williams & Wilkins, can have multiple roles as is known in the art. Therefore, classification of excipients above is not to be construed as limiting in any manner.

In any dosage form, the N-coumaroyldopamine and COMT inhibitor can be administered individually, combined, or as part of a multi-ingredient formula. Individual or combined compositions can be administered one or multiple times per day. Depending on the dosage form, effective daily doses (amounts of each in a given composition) range from about 1 mg each of N-coumaroyldopamine and a COMT inhibitor to about 500 mg each of N-coumaroyldopamine and a COMT inhibitor. In another aspect, the effective daily doses range from about 5 mg each of N-coumaroyldopamine and a COMT inhibitor to about 400 mg each of N-coumaroyldopamine and a COMT inhibitor. Yet, another aspect, the effective daily doses range from about 10 mg each of N-coumaroyldopamine and a COMT inhibitor to about 300 mg each of N-coumaroyldopamine and a COMT inhibitor. Still yet, in another aspect, the N-coumaroyldopamine and the COMT inhibitors are each present in the composition in a range between about 20 mg and about 30 mg. In one aspect, the N-coumaroyldopamine and the COMT inhibitors are each present in the composition in any range between about 5 mg, 10 mg, 15 mg, 20 mg, 25 mg, 30 mg, 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, and 300 mg. Thus, the amounts of the foregoing daily doses can be used in any composition described herein.

In another aspect, EGCG (at least about 50% pure) and N-coumaroyldopamine (at least about 98% pure) can be delivered in daily doses ranging from about 1 mg each of EGCG and N-coumaroyldopamine to about 500 mg each of EGCG and N-coumaroyldopamine. In another aspect, EGCG (at least about 50% pure) and N-coumaroyldopamine (at least about 98% pure) can be delivered in daily doses ranging from about 10 mg each of EGCG and N-coumaroyldopamine to about 300 mg each of EGCG and N-coumaroyldopamine.

Any additional active ingredients can be added to the inventive composition in any amount necessary to accomplish the desired effect. Non-limiting examples of additional active ingredients include agents which increase cAMP and act as a nervous system stimulants (for example, caffeine anhydrous), agents which release norepinephrine and dopamine (for example, phenylethylamine hydrochloride), agents which act as a diuretics and/or cardiac stimulants (for example, theobromine), agents which act as central nervous system stimulants and/or alpha-2-adrenergic antagonists (for example, rauwalscine), agents which act as a beta-2 and beta-3 adrenergic agonists (for example, synephrine), agents which act as monoamine oxidase (MAO) inhibitors (for example, hordenine HCl), or agents which increase carnosine levels in the body to increase endurance (for example, beta-alanine).

An N-coumaroyldopamine and COMT inhibitor composition can be used to aid in bronchodilation, promote weight loss, and improve central nervous system stimulation by increasing the production of cAMP. In another aspect, an N-coumaroyldopamine and EGCG composition can be used as a supplement to increase the bioavailability of N-coumaroyldopamine to increase body fat metabolism, maintain a desired weight, increase energy, improve stamina, increase aerobic capacity, and improve exercise performance.

The present invention overcomes the short half-life of N-coumaroyldopamine by inhibiting its route of metabolism, which involves COMT. By combining N-coumaroyldopamine with a COMT inhibitor, such as EGCG, the half-life of N-coumaroyldopamine is increased. In another aspect, the present invention relates to the manufacture of a composition including N-coumaroyldopamine and a COMT inhibitor, wherein the half-life of N-coumaroyldopamine is increased. In one aspect, a method of making a composition to facilitate body fat mobilization, weight loss, or exercise performance in a user includes combining an effective amount of N-coumaroyldopamine with a catechol-o-methyltransferase inhibitor as described above.

### EXAMPLES

### Constructive Example 1

A synthetic route to make N-coumaroyldopamine, which can be employed in the present invention, includes adding 70 g of p-coumaric acid (2), 99.9 g of dopamine hydrobromide (1), 74.9 g of hydroxybenzotriazole (HOBt), and 700 mL of dimethylformamide (DMF) to a three-neck flask. The mixture is cooled to 0° C., and then 106.4 g of 1-ethyl-3-(3-dimethylaminopropyl)carbodiimide (EDCI) is added drop-wise. Finally, 165.5 g of N,N-diisopropylethylamine (DIPEA) is added. The mixture is stirred at room temperature overnight. To the reaction mixture, 2.8 L H₂O is added, neutralized with aqueous HCl to pH 1, and then extracted with ethyl acetate. The organic layers are combined, dried over Na₂SO₄, and concentrated to give a crude product which can be purified by crystallization in DMF/water to yield 87 g of pure product as an off-white powder.

### Example 2

A N-coumaroyldopamine and EGCG composition was prepared as a capsule formulation. Twenty-five mg N-coumaroyldopamine (about 98% pure) was combined with 75 mg green tea extract (including about 60% EGCG by weight). Additional ingredients included: 75 mg thiamine disulfide butyrate (sulbutiamine), 200 mg 1,3,7-trimethylxanthine (caffeine), 25 mg synephrine hydrochloride, 25 mg theobromine (99% by weight), 25 mg R-methyl-beta-phenylethylamine hydrochloride, 50 mg beta-phenylethylamine hydrochloride, 25 mg hordenine hydrochloride, 30 mg schizanadrol-A (CS), 100 mg blueberry powder extract. Additional excipients included maltodextrin, gelatin, silica, magnesium stearate, and titanium dioxide.

### Example 3

A N-coumaroyldopamine and EGCG composition was prepared as a capsule formulation. Twenty-five mg N-coumaroyldopamine (having a % purity of at least 92%) was combined with 75 mg green tea extract (including about 60% EGCG by weight). Additional ingredients included: 200 mg caffeine anhydrous, SIPERNAT 505 (cellulose derivative, commercially available from Evonik Industries, Parsippany, N.J.), 50 mg phenylethylamine hydrochloride, 25 mg theobromine, 25 mg hordenine HCl, 25 mg ADVANTRA Z (50% pure citrus aurantium extract, commercially available from Nutratech, Inc., West Caldwell, N.J.), 75 mg sulbutiamine, 30 mg schizandrol A, 100 mg blueberry powder extract, and 2.5 mg rauwolscine (90% pure).

### Example 4

A N-coumaroyldopamine and EGCG composition was prepared as a powder formulation. Twenty-five mg of N-coumaroyldopamine was combined with 100 mg green tea extract (98% polyphenols/60% EGCG) by weight). Additional ingredients included 300 mg caffeine anhydrous, 25 mg synephrine, 4 mg rauwolscine (90% pure), 100 mg phenethylamine (PEA), 50 mg hordenine HCl, and 2,000 mg beta-alanine.

With respect to the above description, it is to be realized that the optimum composition for the parts of the invention, to include variations in components, compounds, materials, and use, are deemed readily apparent and obvious to one skilled in the art, and all equivalent relationships to those described in the specification are intended to be encompassed by the present invention.

Therefore, the described aspects are considered as illustrative only of the principles of the invention. Further, various modifications can be made of the invention without departing from the scope thereof, and it is desired, therefore, that only such limitations shall be placed thereon as are imposed by the prior art and which are set forth in the appended claims.

What is claimed is:

1. A composition comprising:
N-coumaroyldopamine; and
a catechol-o-methyltransferase (COMT) inhibitor.

2. The composition according to claim 1, wherein the N-coumaroyldopamine is present in a range between about 10 and about 300 milligrams (mg).

3. The composition according to claim 1, wherein the COMT inhibitor is present in a range between about 10 and about 300 mg.

4. The composition according to claim 1, wherein the N-coumaroyldopamine is at least about 98% pure.

5. The composition according to claim 1, wherein the N-coumaroyldopamine is between about 80% and about 99% pure.

6. The composition according to claim 1, wherein the COMT inhibitor is epigallocatechin gallate.

7. The composition according to claim 1, wherein the COMT inhibitor is a catechin.

8. The composition according to claim 1, wherein the COMT inhibitor is quercetin, harmine, rutin, caffeic acid, chlorogenic acid, rosmarinic acid, or any combination thereof.

9. The composition according to claim 1, wherein the COMT inhibitor is at least about 50% pure.

10. The composition according to claim 1, wherein the COMT inhibitor is between about 40% about 60% pure.

11. The composition according to claim 1, further comprising one or more excipients.

12. The composition according to claim 11, wherein the COMT inhibitor is epigallocatechin gallate.

13. The composition according to claim 11, wherein the composition is formulated as a pill, a capsule, a powder, a drink mix, a beverage, a confectionary syrup, or a chewable tablet.

14. The composition according to claim 11, wherein the N-coumaroyldopamine is present in a range between about 10 mg and about 300 mg.

15. The composition according to claim 13, wherein the N-coumaroyldopamine is present in a range between about 20 mg and about 30 mg.

16. The composition according to claim 12, wherein the epigallocatechin gallate is present in a range between about 10 mg and about 300 mg.

17. The composition according to claim 11, further comprising caffeine anhydrous, phenylethylamine hydrochloride, theobromine, hordinine HCl, citrus aurantium extract, sulbutiamine, or any combination thereof.

18. The composition according to claim 11, further comprising beta-alanine.

19. A method to facilitate body fat mobilization, weight loss, or exercise performance in a user, the method comprising:
administering a composition comprising N-coumaroyldopamine and a COMT inhibitor in a physiologically effective amount to decrease body fat, enhance weight loss, or improve exercise performance.

20. The method according to claim 19, wherein the COMT inhibitor is epigallocatechin gallate.

21. The method according to claim 18, wherein the COMT inhibitor is quercetin, harmine, rutin, caffeic acid, chlorogenic acid, rosmarinic acid, or any combination thereof.
